(19) United States
(12) Patent Application Publication
(10) Pub. No.: US 2026/0319256 A1
(43) Pub. Date: Oct. 15, 2026

(54) VIEWING APPARATUS

(71) Applicant: GENSIGHT BIOLOGICS, Paris (FR)

(72) Inventor: Olivier VILCOT, Paris (FR)

(21) Appl. No.: 19/481,114

(22) PCT Filed: May 3, 2024
(86) PCT No.: PCT/EP2024/062276

§ 371 Date: Nov. 3, 2025

(30) Foreign Application Priority Data
May 4, 2023 (EP) 23315159.6

(51) Int. Cl. G02B 27/01; G02B 26/08

(52) U.S. Cl. CPC G02B 27/0172; G02B 26/0833; G02B 2027/0138; G02B 2027/014

(57) ABSTRACT

The invention concerns a viewing apparatus (10) comprising: —an acquisition unit (16) acquiring an image of the environment and comprising pixels, a reference horizontal line being defined for the acquired image, —a display (17) comprising several pixels, the pixels being arranged so as to form a rectangle for which a main axis is defined, each pixel having a diagonal parallel to the main axis —a processing unit (18) commanding a line of display sub-areas to form an image the reference horizontal line, the line of display sub-areas forming an angle of 45° with the main axis, the display (17) and the projecting unit (18) being spatially arranged so that the image of the line of display sub-areas in the projected image be aligned with an horizontal line of the projected image with a tolerance of 0.5°.

### TECHNICAL FIELD OF THE INVENTION

The present invention concerns a viewing apparatus.

### BACKGROUND OF THE INVENTION

The retina is composed of photoreceptors, which are highly specialized neurons that are responsible for photosensitivity of the retina by phototransduction, i.e. the conversion of light into electrical and chemical signals that propagate a cascade of events within the visual system, ultimately generating a representation of world. In the vertebrate retina, phototransduction is initiated by activation of light-sensitive receptor protein, rhodopsin.

Photoreceptor loss or degeneration, such as in case of retinitis pigmentosa (RP) or macular degeneration (MD), severely compromises, if not completely inhibits, phototransduction of visual information within the retina. Loss of photoreceptor cells and/or loss of a photoreceptor cell function are the primary causes of diminished visual acuity, diminished light sensitivity, and blindness.

Several therapeutic approaches dedicated to retinal degenerative diseases are currently in development, including gene therapy, stem cell therapy, optogenetics, and retinal prostheses.

For example it has been proposed to restore at least partially vision in these patients by modifying a retinal area with visual prosthesis systems. These systems are comprising a retina implant and are helpful tools for at least partially re-establishing a modest visual perception and a sense of orientation for blind and visually impaired users by exploiting said fact that although parts of the retinal tissue have degenerated most of the retina may remain intact and may still be stimulated directly by light dependent electrical stimuli. Typically, retina implant is implanted into the patient's eye, effecting electrical excitation of the remaining neuronal cells upon light stimulation. When being stimulated, these remaining neuronal cells convey the artificially induced electrical impulses to the visual part of the brain through the optic nerve.

Retinal implants can be broadly divided into two categories: epi- and sub-retinal. Epi-retinal devices are placed on or near the inner surface of the retina, i.e. the side of the retina which is first exposed to incident light and along which the nerve fibers of the ganglion cells pass on their way to the optic nerve. Epi-retinal implants typically comprise a chip with a plurality of pixel elements capable of receiving an image projected by an extraocular device (typically a camera and a microelectronic circuit for decoding incident light) on the retina through the lens of the eye, for converting the image into electrical signals and for further conveying the signals into electrical stimuli via a plurality of stimulation electrodes to stimulate the retinal cells adjacent the chip, in order to reconstruct or improve vision of blind or partially blind patients. In contrast, sub-retinal devices are placed under the retina, between the retina and the underlying retinal pigment epithelium or other deeper tissues. Currently available sub-retinal technologies rely on the implantation of a single, rigid and typically planar chip. It has been further shown that it is desirable to be able to implant more than one chip in order to cover a large visual field.

An alternative approach is to restore photosensitivity of the retina of a subject by modifying a retinal area with by optogenetic approaches. Optogenetics is based in combining techniques from optic and genetics to control and monitor cell activities. It consists in (i) genetically modifying target cells in order to render them sensitive to light by the expression of exogenous photoreactive proteins in cellular membrane and (ii) providing illuminating device able to provide light to said photoreactive proteins.

It is an extremely powerful tool for selective neuronal activation/inhibition which can, for example, be used to restore neural functions in living animals, including humans (Boyden et al., 2005, Nature Neuroscience 8 (9): 1263-68), particularly in the eye (Busskamp et al., 2012, Gene Therapy 19 (2): 169-75).

It has been shown that selected wavelengths of light shall be close to the optimal wavelengths of the photoreactive proteins (Nagel et al. 2003, Proceedings of the National Academy of Sciences 100 (24): 13940-45, Klapoetke et al. 2014, Nature Methods 11 (3): 338-46) and that these proteins might have a very low sensitivity to light (Asrican et al. 2013, Front Neural Circuits, 2013, 7:160; Busskamp et al. 2012, Gene Therapy 19 (2): 169-75). In both approaches, retinal prostheses and optogenetic, in order to obtain minimum level of protein or implant activation by light, the intensity of light received by the modified retina (e.g. implant or target cell or protein) shall be above a minimum value (Barrett et al., 2014, Visual Neuroscience 31 (4-5): 345-354).

However, while sufficient light must arrive at modified retina (e.g. at the photoreactive proteins) to provide its activation, it is required to minimize tissue or cell heat and phototoxicity (Yan et al. 2016, Vision Research 121: 57-71). It is further desired to guarantee that the light dose for a given period of time will not cause any tissue or cell damages. Photobiological and ophthalmologic standards set thresholds for the intensity of the source and for the dose received on target are known in the art (see for example ISO 15004-2 2016; “ISO 62471:2006” 2016; § 8.3 of “ANSI Z136 Standards—LIA” 2014). Intensity is converted to irradiance level on target (in mW/mm² or photons·cm⁻²·s⁻¹) from the etendue of the source and surface of the target and dose in a given period of time is defined as the integral of the irradiance over the period of time (mJ/mm² or photons·cm⁻²).

Thus, it is desirable to provide illuminating device able to control intensity of light emitted and guaranteeing that the corresponding dose shall not exceed a maximum value.

Additionally there might be variability among patients in expressing exogenous photoreactive proteins, and threshold for photophobia is highly variable among patients (Hamel 2006, Orphanet Journal of Rare Diseases 1: 40) therefore it might be desirable to be able to adapt the intensity sent to the patient.

Similarly, the eye is itself an optical system with optical aberrations (Navarro, et al. 1998, Journal of the Optical Society of America A 15 (9): 2522), which include aberrations like myopia, hypermyopia and astigmatism. Optical aberrations are also present in emmetropic eyes and are taken into account in photobiological and ophthalmologic standards (ISO 15004-2, 2007; ISO 62471, 2006). These aberrations might reduce the light intensity received by photoactivable proteins, accordingly it might be desirable to provide illuminating device able to at least partially correct these drawbacks.

It is further desirable to provide illuminating device which are miniaturized so that it can be inserted in a device wearable by humans on a daily basis.

Currently available illuminating device to simulate the optogenetic proteins for in vitro experiments have been constructed (Degenaar et al. 2009, Journal of Neural Engineering 6 (3): 35007; Grossman et al. 2010, Journal of Neural Engineering 7 (1): 16004), but they are not miniaturized and are not yet suited for human use.

Head-Mounted displays are used for augmented reality, for virtual reality or for movie display.

However, the light intensity provided by these Head-Mounted Displays is not sufficient and is not configurable to stimulate photoreactive proteins and therefore are not adapted to optogenetics applications.

### SUMMARY OF THE INVENTION

There is therefore a need for a control of a viewing apparatus providing with a better resolution.

To this end, the specification describes a viewing apparatus comprising:
an acquisition unit adapted to acquire an image of the environment, so as to obtain an acquired image, the acquisition unit comprising pixels grouped in respective acquisition sub-areas comprising the same number of pixels, a reference horizontal line being defined for the acquired image,
a display, the display comprising several pixels grouped in respective display sub-areas comprising the same number of pixels,
the pixels being arranged so as to form a rectangle for which a length is defined, each pixel having a diagonal parallel to the length of the rectangle,
each pixel being adapted to be in at least two states, a first state wherein the pixel sends light in a first direction and a second state wherein the pixel does not send light in the first direction,
a processing unit adapted to command the state of each pixel of the display to ensure that the light sent in the first direction conveys the acquired image,
a projecting unit adapted to image the light sent in the first direction by the display to form a projected image on a surface of an object,
the processing unit commanding the state of each display sub-area in a one-to-one relationship with the acquisition sub-areas,
the processing unit commanding a line of display sub-areas to form an image the reference horizontal line, the line of display sub-areas forming an angle of 45° with the main axis of the rectangle, the display and the projecting unit being spatially arranged so that the image of the line of display sub-areas in the projected image be aligned with an horizontal line of the projected image with a tolerance of 0.5°.

According to further aspects of the viewing apparatus, which are advantageous but not compulsory, the viewing apparatus might incorporate one or several of the following features, taken in any technically admissible combination:
each sub-area is formed by only one pixel.
the display comprises an illumination unit and a reflector, the illumination unit being adapted to illuminate the pixels of the reflector and each pixel of the reflector being adapted to send light in a second direction, the first direction being different from the second direction when the reflector is illuminated by light coming from the illumination unit.
the reflector is a digital micromirror device.
the viewing apparatus comprises a unit adapted to modify the state of the display between at least two states, one state being rotated by a rotation of a rotation angle with relation to another state.
the projection unit is adapted to rotate the image of the line of display sub-areas by a predefined angle.
the sum of the rotation angle and the predefined angle is equal to 45°.
the object is a retina of a wearer.

The specification also describes a method for controlling an illumination of a surface of an object by a viewing apparatus, the viewing apparatus comprising:
a display, the display comprising several sub-areas, each sub-area comprising at least one pixel and each pixel being adapted to be in at least two states, a first state wherein the pixel sends light in a first direction and a second state wherein the pixel does not send light in the first direction, and
a projecting unit adapted to image the light sent in the first direction by the display to form a projected image on the surface of the object,

the method being computer implemented and comprising the steps of:
obtaining a value of a radiant flux received by each sub-area of the display, when the display is illuminated by light coming from a light source,
converting each obtained value in an irradiance value, to obtain converted irradiance values,
choosing a mean irradiance value based on the converted irradiance values and a predefined maximum irradiance value, to obtain a target mean irradiance value, and
determining a control law adapted to control independently the state of each pixel of the sub-area over time,
for each sub-area, the control law being such that, during a predefined time duration, the total time duration during which each pixel of the sub-area is in the second state depends upon the ratio of the target mean irradiance value over the obtained value for said sub-area.

According to further aspects of the method for controlling, which are advantageous but not compulsory, the method for controlling might incorporate one or several of the following features, taken in any technically admissible combination:
during the step of determining, the total interval of time during which each pixel is in the second state is proportional to the ratio of the target mean irradiance value over the obtained value for said sub-area.
during the step of determining, the total time duration during which each pixel is in the second state is equal to the product of the predefined time interval with the ratio of the target mean irradiance value over the obtained value for said sub-area.
during a predefined time duration, the time duration during which each pixel is in the second state is continuous.
during a predefined time duration, the predefined time duration is separated in several time interval, the time duration during which each pixel is in the second state being continuous in each interval.
the display comprises an illumination unit and a reflector, the illumination unit being adapted to illuminate the pixels of the reflector and each pixel of the reflector being adapted to send light in a second direction, the first direction being different from the second direction when the reflector is illuminated by light coming from the illumination unit.
each sub-area consists in one pixel.
the object is a retina of a wearer.

The specification describes a method for illuminating a surface of an object by a viewing apparatus, the viewing apparatus comprising:
a display, the display comprising several sub-areas, each sub-area being adapted to be in at least two states, a first state wherein the sub-area sends light in a first direction and a second state wherein the sub-area does not send light in the first direction,
a projecting unit adapted to image the light sent in the first direction by the display to form a projected image on a surface of an object,

the method comprising the steps of:
receiving a control law obtained by a method for controlling as previously described, and
applying the control law to the sub-areas.

According to another aspect, the projecting unit is part of a viewing apparatus, the viewing apparatus further comprising an acquisition unit and a processing unit, the method for illuminating further comprising the steps of acquiring an image of the environment and commanding the display in function of the acquired image.

The specification also concerns a computer program product comprising instructions for carrying out the steps of a method as previously described when said computer program product is executed on a suitable computer device.

The specification also deals with a computer readable medium having encoded thereon a computer program product as previously described.

The specification also deals with a viewing apparatus adapted for illuminating a surface of an object, the viewing apparatus comprising:
a display, the display comprising several sub-areas, each sub-area being adapted to be in at least two states, a first state wherein the sub-area sends light in a first direction and a second state wherein the sub-area does not send light in the first direction,
a projecting unit adapted to image the light sent in the first direction by the display to form a projected image on a surface of an object,

the viewing apparatus being adapted to:
receive a control law obtained by a method for controlling as previously described, and
apply the control law to the sub-areas.

According to further aspects of the viewing apparatus, which are advantageous but not compulsory, the viewing apparatus might incorporate one or several of the following features, taken in any technically admissible combination:
the viewing apparatus further comprises an acquisition unit adapted to acquire an image of the environment, and a processing unit adapted to command the light source in function of the acquired image.
each sub-area is a pixel, the acquisition unit comprising pixels grouped in respective acquisition sub-areas comprising the same number of pixels, a reference horizontal line being defined for the acquired image,
the display comprising several pixels grouped in respective display sub-areas comprising the same number of pixels,
the pixels being arranged so as to form a rectangle for which a length is defined, each pixel having a diagonal parallel to the length of the rectangle,
each pixel being adapted to be in at least two states, a first state wherein the pixel sends light in a first direction and a second state wherein the pixel does not send light in the first direction,
the processing unit being adapted to command the state of each pixel of the display to ensure that the light sent in the first direction conveys the acquired image,
the processing unit commanding the state of each display sub-area in a one-to-one relationship with the acquisition sub-areas,
the processing unit commanding a line of display sub-areas to form an image the reference horizontal line, the line of display sub-areas forming an angle of 45° with the main axis of the rectangle, the display and the projecting unit being spatially arranged so that the image of the line of display sub-areas in the projected image be aligned with an horizontal line of the projected image with a tolerance of 0.5°.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 shows schematically an example of viewing apparatus in a context of use by a wearer, the viewing apparatus comprising a projecting unit,

FIG. 2 illustrates schematically the different elements of the projecting unit of the viewing apparatus of FIG. 1,

FIG. 3 illustrates an example of control law of a component of the projecting unit of FIG. 2,

FIG. 4 illustrates another example of control law of the component of the projecting unit of FIG. 2, and

FIG. 5 illustrates a schematic representation of the effective pixel on a component of the projecting unit in absence and in presence of a specific arrangement for a line of pixels.

### DETAILED DESCRIPTION OF THE PREFERRED EMBODIMENTS

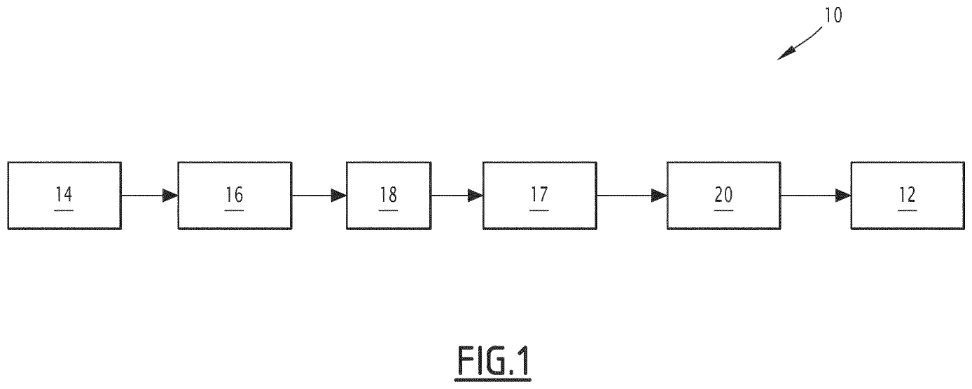

A viewing apparatus 10, a wearer 12 and an environment 14 of the wearer 12 are schematically represented on FIG. 1.

A viewing apparatus 10 is an apparatus used by the wearer 12 of said apparatus for viewing the environment 14.

In the present case, the viewing apparatus 10 is adapted to illuminate the eye, more specifically all or part of the retina, preferably all or part of modified retina, of the wearer 12 with a controlled light intensity.

The light intensity is considered as controlled when the light intensity fulfills a plurality of conditions.

For instance, the plurality of conditions may be chosen among the following ones:
the light intensity at any given time is inferior or equal to a maximum intensity,
the light intensity at any given time be superior or equal to a minimum intensity,
the dose during the period of time be inferior or equal to a maximum value, the period of time being one hour, 12 hours, 24 hours or 48 hours,
an intensity in a given interval of wavelengths,
the illuminated area does not extend beyond a given area, and
several independent spatial areas illuminated by different levels of intensity of light can be defined in a plane corresponding to the retina. One condition is a condition relative to the homogeneity of each elementary level of irradiance. As a specific example, a condition to be fulfilled is that the percentage of variation of level of irradiance with respect to the mean level of irradiance be inferior or equal to a maximum value.

As will appears hereinafter, the viewing apparatus 10 enables to improve the fulfillment of this last condition relative to the homogeneity.

The viewing apparatus 10 comprises an acquisition unit 16, a display 17, a processing unit 18 and a projecting unit 20.

The acquisition unit 16 is adapted to acquire an image of the environment 14.

For instance, the acquisition unit 16 is a camera.

According to a specific embodiment, the camera is an event-based camera. For instance, the acquisition unit 16 is a DAVIS-camera, ATIS-camera or DVS-camera.

In any case, the acquisition unit 16 comprises pixels grouped in respective sub-areas.

Each pixel has here the form of a square.

The display 17 is adapted to display an image.

The display 17 comprises several pixels grouped in respective sub-areas.

Each pixel is adapted to be in at least two positions, a first position wherein the pixel sends light in a first direction and a second position wherein the pixel does not send light in the first direction.

For instance, in this example, the display 17 comprises an optical engine unit 22 and a reflector 23.

The optical engine unit 22 comprises here a light source 24, a fiber 25, an optical system 26 extending from an input 261 to an output 260, a collector 28 and an imaging unit 30.

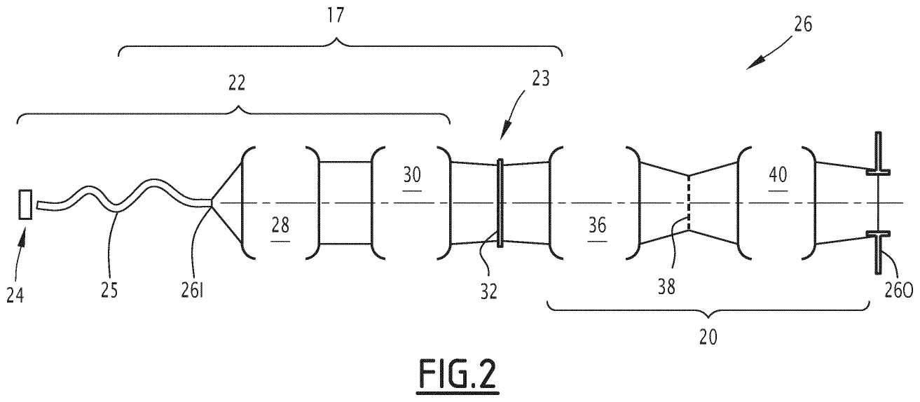

According to the example of FIG. 2, the light source 24 is an electroluminescent diode.

The fiber 25 conveys the light emitted by the light source 24 to the input 261 of the optical system 26.

The present of the fiber 25 permits the light source 24 to be a remote system from the optical system 26.

The collector 28 is adapted to collect the light outputted by the fiber 25.

The collector 28 converts the beam in a collimated beam sent to the imaging unit 30.

The imaging unit 30 is adapted to convert the collimated beam into a beam adapted to illuminate the surface of interest of the reflector 23.

According to the case, the surface of interest is either a part of the surface of the reflector 23 (for instance a disk) or the totality of the surface of the reflector 23

Each pixel of the reflector 23 is adapted to send light in a second direction, the first direction being different from the second direction when the reflector 23 is illuminated by light coming from the optical engine unit 22.

In the present case, the reflector 23 is a digital micromirror device 32.

The digital micromirror device 32 is more often named DMD 32, so that the digital micromirror device 32 will be named hereinafter DMD 32.

This means that each pixel of the DMD 32 is a mirror that can have a first position in which the mirror reflects the light towards the object and a second position in which the mirror reflects the light in another direction.

The processing unit 18 adapted to command the position of each pixel of the display 17 to ensure that the light sent in the first direction conveys the acquired image

In other words, a pixel in the first position means that something has been observed on the acquired image at the same level while a pixel in the second position means that nothing has been observed on the acquired image at the same level.

The DMD 32 is thus used to achieve a digital light processing (also named DLP) and generates an image.

The projecting unit 20 is adapted to project to the retina the image generated by the DMD 32.

The projecting unit 20 comprises a relay unit 36 generating an intermediate image 38 and a projector 40.

The relay unit 36 is adapted to relay the output of the DMD 32 to the input of the projector 40 by generating the intermediate image 38.

This enables to increase the distance between the output of the DMD 32 and the surface while guaranteeing a sufficient field of view.

The projector 40 is adapted to project the intermediate image 38 as a collimated beam.

Each of the collector 28, the imaging unit 30, the relay unit 36 and the projector 40 can be made of one or several lenses and/or mirrors.

The description of the present example does not preclude that the viewing system 10 comprises other elements, such as a compensation lens for near/far-sighted and/or astigmatic users.

For this, the projector 40 is adapted to correct the ametropy of the user by providing an adapted beam, such as a divergent or convergent one.

The operating of the viewing apparatus 10 of FIG. 1 is now described in reference to a method for controlling the illumination to be projected on a retina.

Such method for controlling aims at obtaining a control law adapted to control the illumination sent to the retina of the wearer 12.

Such control ensures a good spatial homogeneity of the light sent to the retina of the wearer 12.

For instance, if the total illuminated surface of the DMD 32 is an area of 30 mm².

For an incident radiant flux of 3 W, the mean irradiance is equal to 100 mW/mm².

However, the irradiance may vary locally from one pixel to another pixel.

More generally, the method also provides with a good homogeneity for any sub-area of the DMD 32.

For instance, instead of one pixel, the method could be applied on sub-areas comprising several pixels, for instance 4 pixels.

The aim of the command law is to alleviate such spatial variation from one pixel to another pixel.

The command law is thus a command law enabling to obtain a spatial homogeneous beam at the level of the retina while keeping the peak irradiance inferior to a predefined maximum irradiance value MIV.

The predefined maximum irradiance value MIV is, for instance, given by a standard fixing the maximum irradiance value for a human eye.

The method for controlling comprises a step of obtaining, a step of converting, a step of choosing, a step of determining and a step of applying.

During the step of obtaining, the value of the radiant flux F received by each pixel of the DMD is obtained.

The value of the radiant flux is obtained by an optical power meter. The probe surface covers the whole beam section at the position of the exit pupil plane of the system.

The relative spatial distribution of the radiant flux is obtained by an external system capable of acquiring an image of the surface of the DMD. For example, a camera with its objective.

Given the total radiant flux value and the relative spatial distribution of the radiant flux, it is possible to retrieve the absolute radiant flux distribution. From the pixel size of the DMD 32, it is possible to retrieve the absolute spatial distribution of the irradiance.

Alternatively, the step of obtaining may be carried out in real-time.

As a specific illustration, one may consider a case wherein the value of the radiant flux F(P1) for a first pixel P1 is equal to 12.5 μW, the value of the radiant flux F(P2) for a second pixel P2 is equal to 10 μW and the value of the radiant flux F(P3) for a third pixel P3 is equal to 7.5 mW.

During the step of converting, each obtained value is converted into an irradiance value IV at the level of the illuminated surface.

For this, in the present case, the ratio of the obtained value over the surface S of the pixel is calculated.

In other words, the processing unit 18 calculates:

$$IV = \frac{F}{S}$$

In the specific example, assuming that each pixel is a square whose side is equal to 10 μm, this leads to a first converted irradiance value IV(P1) of 125 mW/mm² for the first pixel P1, a second converted irradiance value IV(P2) of 100 mW/mm² for the second pixel P2 and a third converted irradiance value IV(P3) of 75 mW/mm² for the third pixel P3.

At this stage, it can be noticed that, as the mean irradiance is equal to 100 mW/mm², the irradiance values vary of ±25% from the mean.

During the step of converting, a target irradiance value TV is chosen.

The target value TV is superior or equal to the minimum of the converted irradiance values.

For 100% homogeneity, the target value TV must be equal to the minimum of the converted irradiance value. But a higher target value TV may be considered for a higher final irradiance level at the cost of a decreased homogeneity. An acceptable minimum level of final homogeneity would be 85%.

In the case of the three pixels P1, P2 and P3, the processing unit 18 chooses the target value TV as the minimum of each converted irradiance value.

This implies that the target value TV is, here, equal to 75 mW/mm².

During the step of determining, the control law is determined.

The control law is adapted to control the angular position of each pixel of the DMD 32 over time.

The time is here divided in frames, each frames having the same predefined time duration PTD.

So as to ensure that target value TV be obtained for each pixel, the command law ensures that, during a predefined time duration PTD, the total time duration TTD during which each pixel is in the second position is equal to the product of the predefined time duration PTD with the ratio of the target mean irradiance value TV over the obtained irradiance value IV for said pixel.

In other words, the following relation is fulfilled:

$$TTD = P T D * \frac{T V}{IV}$$

Applied to the first pixel P1, this leads to the following relations:

$$\frac{T T D ( P 1 )}{PTD} = \frac{T V}{IV ( P 1 )} = \frac{75 mW / {mm}^{2}}{125 mW / {mm}^{2}} = 60 \%$$

Similarly for the second pixel P2, it derives:

$$\frac{TTD ( P 2 )}{PTD} = \frac{T V}{IV ( P 2 )} = \frac{75 mW / {mm}^{2}}{100 mW / {mm}^{2}} = 75 \%$$

Concerning the third pixel P2, the following equations are obtained:

$$\frac{TTD ( P 3 )}{PTD} = \frac{T V}{IV ( P 3 )} = \frac{75 mW / {mm}^{2}}{75 mW / {mm}^{2}} = 100 \%$$

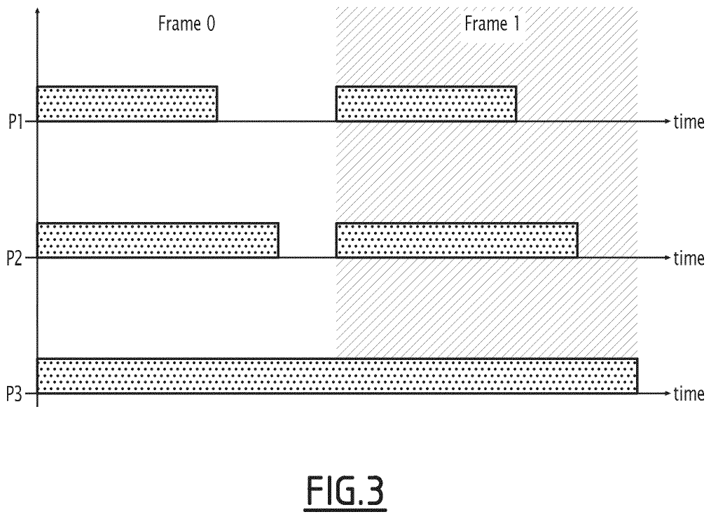

One example of control law fulfilling such criteria is represented on FIG. 3.

FIG. 3 illustrates schematically on two frames how each pixel P1 to P3 is controlled.

For the first pixel P1, starting from 0 to 60% PTD, the first pixel P1 is in the second position while, from 60% to 100% of the PTD, the first pixel P1 is in the first position.

For the second pixel P2, starting from 0 to 75% PTD, the second pixel P2 is in the second position while, from 75% to 100% of the PTD, the second pixel P2 is in the first position.

For the third pixel P3, the third pixel P3 is constantly in the second position.

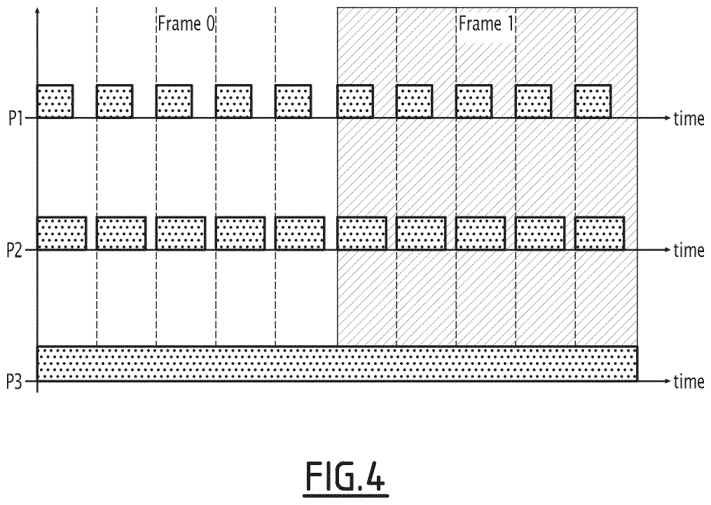

Another example of a control law fulfilling such criteria is represented on FIG. 4.

In this case, the frames are separated in several time intervals and for each time interval, the repartition of FIG. 4 is used.

As a schematic representation, 5 time intervals are used in FIG. 8 and for each of these five time intervals, each pixel are in the second position at the beginning of the time interval and at 60% of the length of the time interval, the first pixel P1 is switched to the first position and similarly at 75% of the length of the time interval, the second pixel P2 is switched to the first position.

Such periodic variation corresponds a pulse width modulation (PWM), whose pulse modulation can vary during a frame.

Many other control laws can be considered.

For instance, one may consider that the pixels with a reduced time in the second position starts in the first position.

For the case of FIG. 4, variants with more or less time intervals can be considered.

In each case, the control law being such, during a predefined time interval, the total interval of time during which each pixel is in the second position depends upon the ratio of the target irradiance value over the obtained value for said sub-area.

Such dependency is here a proportionality.

All the steps that were described before may be considered as a calibration stage and the results are stored in a memory of the processing unit 18.

During the step of applying, the processing unit 18 applies the control law to the DMD 32, and more precisely on each pixel.

In the illustrated case, the mean irradiance provided by the DMD 32 is equal to 75 mW/mm² with a spatial homogeneity of 100%.

The method for controlling therefore enables to obtain a control law adapted to obtain a better homogeneity.

Such better spatial homogeneity is advantageously used in a method for illuminating the retina.

Such method can be used for other devices that a DMD 32.

For instance, the method can be used for a Liquid Crystal on Silicon (LCoS), a LCD (Liquid Cristal Display), a Laser Beam Steering (LBS), an organic light-emitting diode (OLED), an Active-Matrix Organic Light-Emitting Diode (AMOLED) or a micro-LED.

In some of the embodiments, the modification of the pixel is not made in position, the pixel remaining at the same position but rather on whether the pixel is alimented or not.

This means that each pixel has two states which are a first state wherein the sub-area sends light in a first direction and a second state wherein the sub-area does not send light in the first direction.

The change of position is a specific change between two states.

However, it should be pointed out here that there are more than two angular positions, so that there are in fact a plurality of states wherein the sub-area does not send light in the first direction.

The method also provides with a good homogeneity for any sub-area of the DMD 32.

For instance, instead of one pixel, the method could be applied on sub-areas comprising several pixels, for instance 4 pixels.

The sub-areas therefore have at least two states, a first state wherein the sub-area sends light in a first direction and a second state wherein the sub-area does not send light in the first direction.

The first state corresponds to a situation where each pixel sends light in a first direction whereas the second state where each pixel sends light does not send light in the first direction.

The second state is construed here as meaning that each pixel are in the same situation (same position for example), implying that a third state may exist wherein it exists pixels in a situation (for instance not alimented) and pixels in another situation (for instance a specific position).

It may also be considered cases where there are other states for the sub-area corresponding to cases where the sub-area have pixels in both a first state and a second state. This implies that the sub-areas therefore may have more that at least two states.

The method for controlling that has been described so far is implemented by the processor unit.

However, such method can be carried out by any system.

Such system is a computer or computing system, or similar electronic computing device adapted to manipulate and/or transform data represented as physical, such as electronic, quantities within the computing system's registers and/or memories into other data similarly represented as physical quantities within the computing system's memories, registers or other such information storage, transmission or display devices. According to the present invention, those terms are synonyms or equivalents.

Such system may interact with a computer program product to carry out a method for controlling.

In a specific example, the system comprises a processor, a keyboard and a display unit.

According to a variant, the system is a miniaturized computer. The system is for example a miniaturized electronic board containing a processor, memories and fast computing capabilities such as direct memory accesses (whose acronym is DMA).

For example, the electronic board comprises a field-programmable gate array (whose aconym is FPGA), a System On Chip (whose acronym is SoC) or an application-specific integrated circuit (whose acronym is ASIC).

The processor comprises a data-processing unit, memories and a reader. The reader is adapted to read a computer readable medium.

The computer program product comprises a computer readable medium.

The computer readable medium is a medium that can be read by the reader of the processor. The computer readable medium is a medium suitable for storing electronic instructions, and capable of being coupled to a computer system bus.

Such computer readable storage medium is, for instance, a disk, a floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

A computer program is stored in the computer readable storage medium. The computer program comprises one or more stored sequence of program instructions.

The computer program is loadable into the data-processing unit and adapted to cause execution of a method for controlling.

Furthermore, the method can be used for other objects, such as a modified retina, retina cells, modified retina cells or a retina implant.

In particular, the object is a retina of an eye, said retina being modified for expressing plurality of photoreactive proteins.

According to a special embodiment, photoreactive protein is an opsin.

According to preferred embodiment, it is chosen in the group consisting of light-gated ion channel protein, and more specifically it is selected in the group consisting of Chrimson, ChrimsonR, (WO2013/71231), ChrimsonR-tdT, Catch, Channelrhodopsin (US20140121265, U.S. Pat. No. 8,906,360), and melanopsin and their derivatives. According to another special embodiment, photoreactive protein is chosen in the group consisting of light-gated ion pump such as bacteriorhodopsins (Lanyi, J K, 2004, Annu Rev Physiol. 66:665-88), halorhodopsins (Lanyi, J K, 1990, Physiol Rev. 70:319-30), and their derivatives.

Such method can also be advantageously used in combination with a specific arrangement of the display 17 and the projecting unit 20.

To understand the specificity of such arrangement, a reference horizontal line is defined for the acquired image.

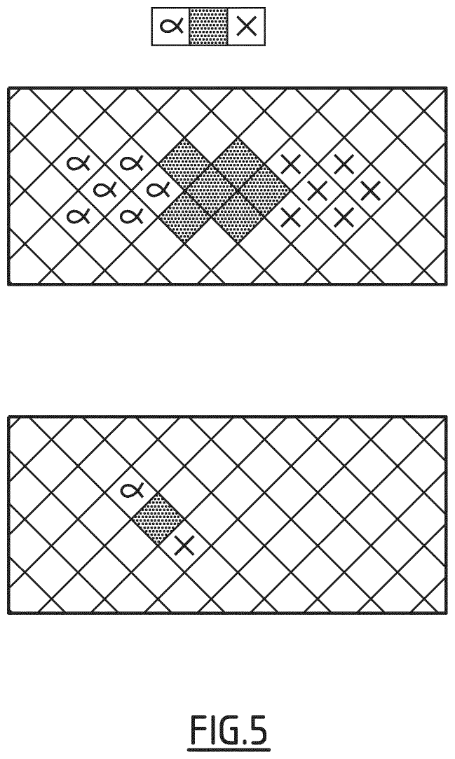

FIG. 5 illustrates schematically how this reference horizontal line is formed on the pixels of the DMD 32 by taking the example of three pixels on the acquisition unit 16 (see above of the figure).

When the specific arrangement is not there, so as to ensure to have a correct image at the output of the projecting unit 20, as represented on the middle part of FIG. 5, one pixel of the acquisition unit 16 corresponds to 6 pixels of the DMD 32 arranged spatially to form a fish form (four pixels for the body of the fish and 2 pixels for the fishtail).

This results in a poor resolution.

By contrast, as represented on the down part of FIG. 5, when the specific arrangement, one pixel of the acquisition unit 16 corresponds to one pixel of the DMD 32.

It can be noted that the pixels of the DMD 32 are arranged so as to form a rectangle with a length, each pixel having a diagonal parallel to the length of the rectangle (the length comprising to the main axis of the rectangle).

This results in an optimal resolution.

More precisely, the processing unit 18 commands a line of pixels of the DMD 32 to form the horizontal reference line and the display 17 and the projecting unit 20 are spatially arranged so that the image of the line of pixels in the projected image be aligned with an horizontal line of the projected image with a tolerance of 0.5°.

In this context, a tolerance is to be understood as meaning that the equality is fulfilled in an interval comprised between the value−the tolerance and the value+the tolerance.

However, imaging such line of pixels with the same projecting unit 20 as in the case of the middle part of FIG. 5 will result in the projected image as a line with an angle of 45° with the horizontal.

This means that the specific arrangement also implies that the orientation of the display 17 and the way the projecting unit 20 the lines are chosen so that the image of the line of pixels be aligned with a horizontal line.

For instance, this can be obtained by using a unit adapted to modify the position of the display 17 between at least two positions, one position being rotated by a rotation of a rotation angle with relation to another position.

In case the rotation angle is equal to 45°, the alignment is obtained.

The display 17 can also be rotated in a fixed manner around its optical axis by the rotation angle.

Alternatively, the projection unit is adapted to rotate the image of the line of pixels by a predefined angle.

In case the predefined angle is equal to 45°, the alignment is obtained.

In variant, both embodiments can be combined provided the sum of the rotation angle and the predefined angle is equal to 45°.

Such specific arrangement enables to obtain a better resolution while keeping a simple set-up.

It can be noticed that such method is also usable with larger sub-areas provided the processing unit 18 commands the position of each sub-area in a one-to-one relationship with the sub-areas of the acquisition unit 16, each sub-area comprising the same number of pixels.

1. A viewing apparatus comprising:
an acquisition unit adapted to acquire an image of the environment, so as to obtain an acquired image, the acquisition unit comprising pixels grouped in respective acquisition sub-areas comprising the same number of pixels, a reference horizontal line being defined for the acquired image,
a display, the display comprising several pixels grouped in respective display sub-areas comprising the same number of pixels,
the pixels being arranged so as to form a rectangle for which a main axis is defined, each pixel having a diagonal parallel to the main axis of the rectangle,
each pixel being adapted to be in at least two states, a first state wherein the pixel sends light in a first direction and a second state wherein the pixel does not send light in the first direction,
a processing unit adapted to command the state of each pixel of the display to ensure that the light sent in the first direction conveys the acquired image, and
a projecting unit adapted to image the light sent in the first direction by the display to form a projected image on a surface of an object,
the processing unit commanding the state of each display sub-area in a one-to-one relationship with the acquisition sub-areas,
the processing unit commanding a line of display sub-areas to form an image the reference horizontal line, the line of display sub-areas forming an angle of 45° with the main axis of the rectangle, the display and the projecting unit being spatially arranged so that the image of the line of display sub-areas in the projected image be aligned with an horizontal line of the projected image with a tolerance of 0.5°.

2. The viewing apparatus according to claim 1, wherein each sub-area is formed by only one pixel.

3. The viewing apparatus according to claim 1, wherein the display comprises an illumination unit and a reflector, the illumination unit being adapted to illuminate the pixels of the reflector and each pixel of the reflector being adapted to send light in a second direction, the first direction being different from the second direction when the reflector is illuminated by light coming from the illumination unit.

4. The viewing apparatus according to claim 3, wherein the reflector is a digital micromirror device.

5. The viewing apparatus according to claim 1, wherein the viewing apparatus comprises a unit adapted to modify the state of the display between at least two states, one state being rotated by a rotation of a rotation angle with relation to another state.

6. The viewing apparatus according to claim 1, wherein the projection unit is adapted to rotate the image of the line of display sub-areas by a predefined angle.

7. The viewing apparatus according to claim 5, wherein the sum of the rotation angle and the predefined angle is equal to 45°.

8. The viewing apparatus according to claim 1, wherein the object is a retina of a wearer.
